(19) United States
(12) Patent Application Publication
(10) Pub. No.: US 2024/0399066 A1
(43) Pub. Date: Dec. 5, 2024

(54) USER INTERFACE MEMBER FOR A DRUG DELIVERY DEVICE AND DRUG DELIVERY DEVICE

(71) Applicant: Sanofi, PARIS (FR)

(72) Inventors: Paul Richard Draper, Warwick (GB); Ronald Anthony Smith, Salir, Loule (PT); Daniel Paul Jenkins, Warwick (GB)

(21) Appl. No.: 18/694,806

(22) PCT Filed: Sep. 22, 2022
(86) PCT No.: PCT/EP2022/076288

§ 371 Date: Mar. 22, 2024

(30) Foreign Application Priority Data
Sep. 24, 2021 (EP) 21315175.6

(51) Int. Cl. A61M 5/315

(52) U.S. Cl. CPC A61M 5/31546; A61M 2205/502; A61M 2205/584; A61M 2205/587

(57) ABSTRACT

The present disclosure relates to a user interface member for a drug delivery device comprising a light-emitting region configured to be illuminated by at least one light-emitting element in order to emit light from the light-emitting region and to visually indicate an operation state of the drug delivery device to a user. The light-emitting region comprises two or more subregions which are illuminable independently of each other by the at least one light-emitting element in order to present different illumination patterns to a user via the light-emitting regions. The different illumination patterns indicate different operation states of the drug delivery device to the user.

### CROSS-REFERENCE TO RELATED APPLICATIONS

The present application is the national stage entry of International Patent Application No. PCT/EP2022/076288, filed on Sep. 22, 2022, and claims priority to Application No. EP 21315175.6, filed on Sep. 24, 2021, the disclosures of which are incorporated herein by reference.

### TECHNICAL FIELD

A user interface member for a drug delivery device is provided. Furthermore, a drug delivery device is provided. Moreover, an operation method for a user interface member is provided.

### BACKGROUND

Administering an injection is a process which presents a number of risks and challenges for users and healthcare professionals, both mental and physical. A drug delivery device may aim to make self-injection easier for patients. Drug delivery devices using electronics are becoming increasingly popular in the pharmaceutical industry as well as for users or patients. Visually indicating different operation states of the drug delivery device may make usage of the drug delivery device easier and more comfortable.

### SUMMARY

One object to be achieved is to provide an improved user interface member for a drug delivery device. Preferably, the user interface member may visually indicate to a user the different operation states of the drug delivery device. Further objects to be achieved are to provide an improved drug delivery device and an improved operation method of a user interface member.

These objects are achieved, inter alia, by the subject-matter of the independent claims. Advantageous embodiments and further developments are subject of the dependent claims and are also presented in the following description and in the figures.

Firstly, the user interface member is specified. The user interface member may be a button and/or a knob for operating or activating the drug delivery device. The user interface member may be connected or connectable to a drug container holder of the drug delivery device. The user interface member may form a proximal end of the drug delivery device.

According to at least one embodiment, the user interface member comprises a light-emitting region configured to be illuminated by at least one light-emitting element in order to emit light from or via the light-emitting region, respectively, e.g. to visually indicate an operation state of the drug delivery device to a user. For example, light from the light-emitting element is directed to the light-emitting region and is then emitted from the user interface member via the light-emitting region.

The light-emitting region may be a continuous region of the user interface member. The light-emitting region may be at least sectionally transparent or translucent, particularly for visible light. In particular, the light-emitting region is transparent or translucent over a large part of its area, e.g. over at least 60% or at least 75% or at least 90% or over 100% of its area. For example, the light-emitting region is at least sectionally, particularly completely, formed from a transparent or translucent material, e.g. a plastic. The light-emitting region may be ring-like.

The light-emitting region may be formed continuously transparent or translucent. Alternatively, the light-emitting region may comprise two or more transparent or translucent subregions which are separated from each other, e.g. by one or more opaque regions.

The light-emitting region may, in particular, be a region of an outer surface of the user interface member, e.g. a region which can be physically touched by a user and/or viewed from outside. In other word, the light-emitting region may be formed by a light-emitting surface which is a partial surface of the outer surface of the user interface member.

The light-emitting region may be the only region of the user interface member transparent or translucent to visible light. Other regions of the user interface member, particularly regions of the outer surface of the user interface member adjoining the light-emitting region, may be opaque, i.e. not transparent or translucent for visible light. During operation of the user interface member, light from the at least one light-emitting element is preferably only emitted via the light-emitting region.

“At least one light-emitting element” may include exactly one or more, e.g. exactly two or more light-emitting elements.

According to at least one embodiment, the light-emitting region comprises two or more subregions. The subregions may be illuminable independently of each other by the at least one light-emitting element in order to present different illumination patterns to a user via the light-emitting region. The subregions may be separated from each other, e.g. by opaque regions or regions not illuminated, or may directly adjoin each other. Each subregion may be transparent or translucent over its entire area, e.g. completely formed from a transparent or translucent material. During operation, light may be emitted from the user interface member via each of the subregions. For example, an illuminated subregion emits light over its entire area.

According to at least one embodiment, the different illumination patterns indicate one or more operation states of the drug delivery device to the user, e.g. different operation states. For example, the drug delivery device has two or more operation states. Different operation states may be assigned different illumination patterns, e.g. on a one-to-one basis, i.e., each state may have a unique illumination pattern assigned to it.

For example, the operation states may comprise a first operation state or pairing state. In this state, the device may attempt to perform (e.g. advertise for) and/or may perform a wireless pairing procedure. The wireless pairing procedure may be configured to pair the drug delivery device or the user interface member with a further device, e.g. for creating a secured or protected wireless connection between the further device and the drug delivery device or the user interface member. Once the pairing procedure has been completed, the further device is paired with the user interface member or the drug delivery device. The pairing may be temporary, e.g. for only one communication session or permanent, e.g. for a number of separate communication sessions. Paired electronic elements (e.g. the further device and the user interface member or the drug delivery device) expediently can establish a secure communication channel without going through the pairing procedure again, especially when the elements are both operational and one element attempts to connect to the other one. During the pairing procedure, keys for encrypting data sent through the channel may be sent from one electronic element to the other electronic element and vice versa. When it is advertising for pairing or attempting to pair, the electronic element may signal that it is ready to be paired with other electronic elements, e.g. in its proximity. The pairing procedure may be a Bluetooth pairing procedure. Between the paired electronic elements a Bluetooth pairing may be established. Data transmission between the user interface member or the drug delivery device and the further device, e.g. dose data or information from the user interface member or the drug delivery device to the further device, may be restricted to further devices which are paired to the user interface member or the drug delivery device.

Alternatively or additionally, the operation states may comprise a second operation state or transmission or synchronization state. In this state, the drug delivery device or the user interface member may transmit, e.g. synchronize, or attempt to transmit, e.g. synchronize, data to a further device, e.g. the further device which is paired with the drug delivery device or the user interface member. At first the drug delivery device or the user interface member may advertise that it intends to transmit data, e.g. dose or dose history data. If the further device is within reach of the communication interface, the (secure) communication channel is established and the data may be transmitted, e.g. synchronized. The data may be dose date, e.g. dose history data.

The illumination pattern for the respective state may indicate, that the drug delivery device or the user interface member is advertising/attempting to pair or transmit. When the pairing process or the transmission process is running, the light-emitting element may not emit light. This saves electrical power.

The further device may be a computer, a smart phone, or a smart watch, for example. The user interface member or the drug delivery device may comprise a wireless communication interface, e.g. a Bluetooth communication interface. This wireless communication interface may be configured to communicate with the further device. Alternatively or additionally, the operation states may comprise a third operation state or warning state, e.g. a state in which the power stored in a power source of the user interface member or the drug delivery device is near its end. Alternatively or additionally, the operation states may comprise a fourth operation state or dose dial state, in which a drug dose is dialed, and/or a fifth operation state or dose delivery state in which a drug dose is delivered, e.g. injected.

In at least one embodiment, the user interface member for a drug delivery device comprises a light-emitting region configured to be illuminated by at least one light-emitting element in order to emit light from the light-emitting region and to visually indicate an operation state of the drug delivery device to a user. The light-emitting region comprises two or more subregions which are illuminable independently of each other by the at least one light-emitting element in order to present different illumination patterns to a user via the light-emitting regions. The different illumination patterns indicate different operation states of the drug delivery device to the user.

Light-emitting elements, like LEDs, may be used in electronic devices to indicate states, e.g. on/off states or a standby state, to a user. Sometimes, color is incorporated, for instance green to indicate ready and red to indicate not ready, whilst text and/or symbols adjacent to the LEDs provide may further information.

As a medical device, regulations and standards reserve the use of certain colors for defined states, or prevent the use of other colors for reasons of patient safety. Furthermore, colorblindness amongst a proportion of the user population may lead to confusion if certain color combinations are used.

With the user interface member specified herein, it is possible to communicate the operation states of the drug delivery device by means other than color, namely by different illumination patterns. This is achieved by using a light-emitting region with two or more subregions which can be illuminated independently of each other.

The user interface member and/or the drug delivery device specified herein may be elongated and/or may comprise a longitudinal axis, e.g. a main extension axis. Additionally or alternatively, the user interface member and/or the drug delivery device may have a rotational symmetry with respect to the longitudinal axis. A direction parallel to the longitudinal axis is herein called an axial direction. By way of example, the drug delivery device and/or the user interface member may be cylindrically-shaped.

Furthermore, the drug delivery device may comprise an end, e.g. a longitudinal end, which may be provided to face or to be pressed against a skin region of a human body. This end is herein called the distal end. A drug or medicament may be supplied via the distal end. The opposite end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device, e.g. of the user interface member, is herein understood to be the end of the member/element/feature located most distally. Accordingly, the proximal end of a member or element or feature is herein understood to be the end of the element/member/feature located most proximally.

In other words, distally is used herein to specify directions, ends or surfaces which are arranged or are to be arranged to face or point towards a dispensing end of the drug delivery device or components thereof and/or point away from, are to be arranged to face away from or face away from the proximal end. On the other hand, proximal is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end and a distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis. The term “angular direction”, “azimuthal direction” or “rotational direction” are herein used as synonyms. Such a direction is a direction perpendicular to the longitudinal axis and perpendicular to the radial direction.

According to at least one embodiment, the user interface member comprises the at least one light-emitting element. The light-emitting element may be a light-emitting diode, LED for short. Other light-emitting elements are possible, e.g. electroluminescent elements such as luminescent foils. The light-emitting element may be configured to emit white light. Particularly, the user interface member may be configured such that the light generated by the light-emitting element is directed onto the light-emitting region in order to emit the light via the light-emitting region.

The user interface member may further comprise an energy source, e.g. a cell or battery, for powering the at least one light-emitting element. The at least one light-emitting element may be mounted on a carrier, e.g. a PCB, like a flexible PCB. The light-emitting element may be electrically connected via the carrier.

According to at least one embodiment, the user interface member is configured to operate the at least one light-emitting element depending on the operation state of the drug delivery device in order to present different illumination patterns to a user via the light-emitting region. For example, the user interface member is configured to operate the light-emitting element in a flashing mode and/or in a continuous mode.

For operating the light-emitting element, the user interface member may comprise a control unit which is configured to control the light-emitting element. The control unit may be mounted on the same carrier as the light-emitting element.

According to at least one embodiment, the light-emitting element is a side looker. A side looker is a light-emitting element which emits light essentially parallel to a mounting surface on which the light-emitting LED is mounted and/or a light-emitting element with a radiation-exit side running obliquely or perpendicularly to the mounting surface.

According to at least one embodiment, the user interface member comprises two or more light-emitting elements. E.g., the user interface member comprises exactly two light-emitting elements. All features disclosed herein for one light-emitting element are also disclosed for the other light-emitting element(s). Particularly, all light-emitting elements may be LEDs, e.g. side looker LEDs.

According to at least one embodiment, the light-emitting elements emit light of the same color. E.g., the light-emitting elements emit white light.

According to at least one embodiment, each light-emitting element is assigned to one subregion. Preferably, each light-emitting element is assigned to one subregion on a one-to-one basis. For example, a large part of light generated by a light-emitting element, e.g. at least 75% or at least 90% of the generated light or all of the generated light, is directed to the assigned subregion and is then emitted from the user interface member via the assigned subregion.

According to at least one embodiment, the subregions of the light-emitting region are arranged to emit light in different, particularly in opposite, directions. For example, the subregions are arranged to emit light in opposite radial directions. For example, the subregions are arranged on different or opposite sides of the user interface member. During operation of the user interface member, light emitted from the different subregions may therefore be emitted in different or opposite directions, particularly in opposite radial direction.

According to at least one embodiment, the two light-emitting elements are arranged such that during operation, they emit light in different, e.g. opposite, directions. For example, the light-emitting elements are arranged to emit light in opposite radial directions.

According to at least one embodiment, the user interface member is configured to operate the light-emitting elements depending on the operation state of the drug delivery device in order to present different illuminations patterns to a user via the light-emitting region. For example, the user interface member is configured to operate the different light-emitting elements individually and/or independently of each other. For example, the user interface member is configured to operate each of the light-emitting elements in a flashing mode and/or continuous mode.

According to at least one embodiment, the different illumination patterns comprise one or more of: a first illumination pattern, a second illumination pattern, a third illumination pattern and a fourth illumination pattern.

According to at least one embodiment, the first illumination pattern is an illumination pattern in which light is emitted simultaneously from at least two, e.g. from all, different subregions in a continuous mode or flashing mode for a predetermined time.

A continuous mode herein means that light is emitted from the respective subregion such that it appears to a user that light is emitted continuously during the predetermined time or a certain time window, i.e. emitted without interruptions. A flashing mode is herein understood to be a mode in which light flashes are periodically emitted during the predetermined time. For example, at least two flashes or at least five flashes are emitted during the predetermined time. Between two light flashes, no light or less light is emitted from the respective subregion such that a user notices this. For example, in the flashing mode, a flash is less than 1 second, e.g. 100 ms, long in time. For example, in the flashing mode, the frequency of the appearance of light flashes is at least 0.5 Hz, e.g. 1 Hz.

According to at least one embodiment, the second illumination pattern is an illumination pattern in which light is emitted in an alternating manner from at least two different subregions for a predetermined time. This means, a first subregion emits light, e.g. in continuous mode or a flashing mode, for a certain time window, e.g. of less than 2 seconds or less than 1 second, e.g. 100 ms, during which at least another subregion does not emit light or emits less light. After that the another subregion emits light for a certain time window, e.g. in continuous or flashing mode, and the first subregion does not emit light or emits less light during this time window. Alternation takes place, e.g., with a frequency of at least 0.5 Hz.

According to at least one embodiment, a third illumination pattern is an illumination pattern in which light is emitted from one subregion for a predetermined time, e.g. in continuous mode or flashing mode, while another subregion does not emit light or emits less light during the predetermined time. For example, the other subregion(s) is (are) turned off for the whole predetermined time.

According to at least one embodiment, the fourth illumination pattern is an illumination pattern in which light of different brightness is emitted from the different subregions for a predetermined time. For example, a first subregions emits light with a higher brightness than a second subregion, e.g. with a brightness which is at least double the brightness of the second subregion, during the predetermined time.

The predetermined time may be, e.g., at least 0.5 seconds or at least one second or at least two seconds. Additionally or alternatively, the predetermined time may be at most 20 seconds or at most 10 seconds or at most 5 seconds.

Other illumination patterns may be realized as well. Usage of more subregions and/or more light-emitting elements allows more illumination patterns.

According to at least one embodiment, the user interface member is configured to present two or more or each of the mentioned illumination patterns. Each of the different illumination patterns may be assigned a different operation state of the drug delivery device, e.g. on a one-to-one basis.

According to at least one embodiment, the user interface member has a cylindrical shape. Particularly, an outer surface for gripping or manipulating the user interface member, e.g. for dose setting or dialing, may have a cylindrical shape. Additionally or alternatively, the user interface member may have a rotational symmetry with respect to the longitudinal axis.

According to at least one embodiment, the user interface member has a lateral surface. The lateral surface may be an outer surface configured to be touched by a user. The lateral surface may delimit the user interface member in outward radial direction. Particularly, the lateral surface may run parallel or acute-angled to the longitudinal axis. The lateral surface may be provided to be touched for a dose setting operation.

The lateral surface may be configured to be held by a user using two fingers for performing a rotation of the user interface member around the longitudinal axis.

Alternatively or additionally, the user interface member may furthermore comprise a proximal surface facing in proximal direction. The proximal surface may run perpendicularly or obliquely with respect to the longitudinal axis and/or the lateral surface. The proximal surface may be configured to be touched by the user, e.g. using only one finger such as the thumb. The proximal surface may be configured for pushing the user interface member in distal direction, e.g. by the user touching this surface. The proximal surface may be provide to be touched for a dose delivery operation.

According to at least one embodiment, the lateral surface may comprise gripping features, e.g. grooves. The grooves may extend parallel or acute-angled to the longitudinal axis. The gripping features may simplify gripping of and/or manipulating the user interface member by a user.

According to at least one embodiment, the light-emitting region extends circumferentially at an outer surface of the user interface member. The light-emitting region may be formed in the lateral surface of the user interface member or may adjoin the lateral surface.

“Extend circumferentially” may mean, in particular, that the light-emitting region extends around, e.g. completely around, the longitudinal axis. E.g. the light-emitting regions forms a closed loop around the longitudinal axis.

According to at least one embodiment, the light-emitting region forms an edge between the lateral surface and the proximal surface of the user interface member. For example, the light-emitting region forms a transition region between the lateral surface and the proximal surface.

According to at least one embodiment, the light-emitting surface, which forms the light-emitting region, runs obliquely to the longitudinal axis and/or to the lateral surface and/or to the proximal surface. For example, an angle between the light-emitting surface and the longitudinal axis and/or the lateral surface and/or the proximal surface is at least 10° or at least 20° or at least 30°. Additionally or alternatively, the angle may be at most 80° or at most 70° or at most 60°.

According to at least one embodiment, the light-emitting region or the light-emitting surface forming the light-emitting region, respectively, has a linear shape. This means, a length of the light-emitting region is much greater than a width of the light-emitting region, e.g. at least 5 times or at least 10 times the width. E.g., the width of the light-emitting region is at most 5 mm or at most 2 mm.

The light-emitting region may, in particular, have a curved linear shape and/or a shape with changing extension direction. For example, the light-emitting region is ring-shaped. The subregions may be formed as ring segments.

According to at least one embodiment, the user interface member further comprises a light distribution element. The light distribution element may have a light-entry side and a light-exit side. The light distribution element may be configured to receive light from the light-emitting element at or via the light-entry side and transfer the light to the light exit-side. The light may leave the light distribution element via the light-exit side. The light-exit side may be assigned to the light-emitting region.

The light distribution element may be a solid body. For example, the light distribution element is formed in one piece or is integrally formed, respectively. The light-entry side and/or the light-exit side may be surfaces of the light distribution element, e.g. opposite surfaces. The light distribution element may be formed of a transparent or translucent material, e.g. of a plastic.

The light-exit side may face the light-emitting region or may form the light-emitting region or the light-emitting surface, respectively. In other words, the light-exit side of the light distribution element may form a part of the outer surface of the user interface member. Particularly, the light-exit side may be ring-shaped and/or may delimit the light distribution element in outward and/or radial direction.

The at least one light-emitting element may be arranged in the user interface member such that the radiation-exit side of the light-emitting element faces the light-entry side of the light distribution element, e.g. such that most of the light emitted by the light-emitting element is coupled into the light distribution element via the light-entry side.

According to at least one embodiment, the user interface member comprises a gripping element. The gripping element may be formed from a plastic. The gripping element may be opaque to the light of the light-emitting elements. The gripping element may form a large part of the lateral surface of the user interface member, e.g. at least 75% or at least 90% of the area of the lateral surface. For example, the gripping element as a hollow cylindrical shape. The gripping element may be formed in one piece.

According to at least one embodiment, the user interface member comprises a cover element. The cover element may be formed in one piece and/or may be opaque to the light of the light-emitting element and/or may be formed of plastic. The cover element may form the proximal surface of the user interface member.

According to at least one embodiment, the light-emitting region is arranged, e.g. in axial direction, between the cover element and the gripping element. Particularly, the light distribution element is arranged, e.g. in axial direction, between the gripping element and the cover element. The light distribution element may be connected to the gripping element in a form-fitting manner and/or adhesive manner. The cover element may be connected to the light distribution element and/or the gripping element in a form-fitting manner and/or adhesive manner. Alternatively two of the components, e.g. the cover element and the light distribution element are twin-shot molded. However, any combination of twin-shot molding, gluing, clipping, welding etc. could be used between any of the three individual components to form the same functional geometry.

According to at least one embodiment, the light distribution element comprises a further surface. The light distribution element may be arranged with respect to the gripping element such that the further surface faces or abuts an inner surface of the gripping element. The inner surface of the gripping element may be a surface facing towards the longitudinal axis or in radial inward direction, respectively. For example, the further surface is covered by the gripping element in radial direction. The further surface may delimit the light distribution element in outward radial direction and/or may be ring-shaped.

According to at least one embodiment, the light distribution element has the shape of a plate. This means, in particular, that the light distribution element is essentially formed as a plate. For example, the light distribution element is disc-shaped.

According to at least one embodiment, the light distribution element has a main extension plane. In the main extension plane a thickness of the light distribution element measured perpendicularly to the main extension plane may be smaller, e.g. at least by a factor of 5 or 10, than extensions of the light distribution element along the main extension plane. The light distribution element may be arranged such that the main extension plane of the light distribution element runs obliquely or perpendicularly to the longitudinal axis and/or to the lateral surface.

According to at least one embodiment, the light-exit side of the light distribution element is formed by a surface of the light distribution element running obliquely or perpendicularly to the main extension plane of the light distribution element. Additionally or alternatively, the light-entry side is formed by a surface of the light distribution element running obliquely or perpendicularly to the main extension plane of the light distribution element. During operation, light may be transferred or guided in the light distribution element from the light-entry side to the light-exit side, e.g. guided parallel or essentially parallel to the main extension plane of the light distribution element.

According to at least one embodiment, the light distribution element has at least two subsections. Each subsection may be assigned to one subregion of the light-emitting region, e.g. on a one-to-one basis. For example, each subsection comprises a part of the light-exit side and this part of the light-exit side faces or forms the assigned subregion. The subsections may be separated from each other by one or more structures, e.g. grooves or recesses, in the light distribution element. For example, the subsections are formed symmetrically with respect to mirroring at the longitudinal axis. The structures may be oriented in radial direction or may extend in radial direction, respectively.

According to at least one embodiment, the two subsections are optically decoupled from each other. For example, light coupled into the light distribution element in one subsection via the light-entry side is prevented from reaching into the other subsection e.g. by means of internal reflections in the light distribution element. For example, the light distribution element comprises one or more reflection surfaces or reflection interfaces for optically decoupling the two subsections. The reflection surfaces or interfaces may extend in radial direction.

According to at least one embodiment, e.g. alternatively or additionally to the structures separating the subsections, the light distribution element may be configured such that light from one light-emitting element is guided, e.g. at least predominantly or entirely, to a first subregion, e.g. a region of the light exit side and/or with an angular extension of more than 120° and/or less than or equal to 180°. Light from a further light-emitting element is guided, e.g. at least predominantly or entirely, to a second subregion, preferably with an angular extension of more than 120° and/or less than or equal to 180°. The first and the second subregion may angularly adjoin each other.

According to at least one embodiment, a recess is formed in the light distribution element. For example, the recess is formed in the center of the light distribution element. The longitudinal axis may run through the recess.

According to at least one embodiment, the recess is a hole. The hole may extend completely through the light distribution element, e.g. in axial direction and/or perpendicularly to the main extension plane of the light distribution element.

According to at least one embodiment, the recess is configured to receive the at least one light-emitting element. In other words, the recess is such large that at least one light-emitting element, preferably two or more light-emitting elements can be placed inside the recess. The light-emitting element(s) of the user interface member may be arranged in the recess.

According to at least one embodiment, the light-entry side adjoins the recess or delimits the recess respectively. E.g., the light-entry side circumferentially surrounds the recess. Particularly, the light-entry side may delimit the recess in outward radial direction.

According to at least one embodiment, the light distribution element is configured to guide light from the light-entry side to the light exit side by reflection and/or refraction. By way of example, the light is guided in the light distribution element by non-imaging optics. For example, each subsection of the light distribution element comprises or is delimited by structures configured to guide the light from the light-entry side towards the assigned subregion by reflection and/or refraction. The structures may be oriented in radial direction or may extend in radial direction, respectively. The structures may be surfaces or interfaces of the light distribution element, e.g. material to air interfaces. The light distribution element may be a plastic component, e.g. a molded plastic component.

According to at least one embodiment, the user interface member is a button and/or knob for a drug delivery device. The user interface member may be configured to be rotated and/or axially moved relative to a housing of the drug delivery device and/or to be pressed. The user interface member may be configured such that by operating it in first way, e.g. by rotating it, a dose dial is initiated or performed and by operating it in second way, e.g. axially moving it, an injection process is initiated or performed. The user interface member may therefore realize a dose dial functionality and an injection functionality.

According to at least one embodiment, the user interface member is configured to be manually operated by a user in order to initiate different operation states of the drug delivery device. For example, the user interface member and/or the drug delivery device are configured to set the operation state depending on time related features of the manual operation. Particularly, different time related features may be assigned different operation states.

For example, the user interface member and/or the drug delivery device are configured such that movement of the user interface member in the same direction and/or by the same distance and/or holding the user interface member in the same position but doing so with different time related features initiates different operation states. The time related features may be, e.g., different lengths of time of operation the user interface member and/or different number of repetitions of operation.

According to at least one embodiment, the user interface member is configured to generate different electronic signals depending on the way the user interface member is operated, e.g. depending on the time related features of operation. Each of the different electronic signals may be assigned to a different operation state. For example, the different electronic signals are generated depending on the time related features of operating the user interface member.

According to at least one embodiment, the different electronic signals are associated with different operation states of the drug delivery device. For example, each electronic signal is associated with a different operation state of the drug delivery device. The different electronic signals may be used to initiate the different operation states of the drug delivery device, e.g. by using a control unit of the user interface member.

According to at least one embodiment, the different operation states are assigned the different illumination patterns. For example, the drug delivery device or the user interface member is configured such that when initiating an operation state, the assigned illumination pattern is generated automatically. This provides the user with the information which operation state he/she has initiated.

In at least one embodiment, the user interface member is configured to be manually operated by user in order to initiate different operation states of the drug delivery device and configured to generate different electronic signals depending on the way the user interface member is operated. The different electronic signals are associated with different operation states of the drug delivery device and the different operation states are assigned to different illumination patterns.

According to at least one embodiment, manual operation of the user interface member comprises at least one of: touching, pressing, rotating. The user interface member may comprise one or more sensors for detecting the manual operation, like a touch sensor, an acceleration sensor, an IR sensor and so on.

Next, the operation method of the user interface member is specified. The user interface member for the operation method may be the user interface member specified above. All features disclosed for the user interface member are therefore also specified for the operation method and vice versa.

In at least one embodiment, the operation method of the user interface member comprises the steps:
generating an electronic or electrical signal depending on the way a user manually operates the user interface member, wherein the electronic signal is associated with an operation state of the drug delivery device,
operating the at least one light-emitting element depending on the operation state and/or the electronic signal in order to present an illumination pattern to a user via the light-emitting region assigned to the operation state.

The electronic signal may also be used to initiate the operation state.

Next, the drug delivery device is specified. The drug delivery device may be an injection device and/or a pen type device, e.g. a dial extension pen. The drug delivery device may be a variable dose device in which the drug dose to be delivered to a user can be variably set. For example, the drug delivery device is a reusable device.

According to at least one embodiment, the drug delivery device comprises a user interface member. The user interface member may, in particular, be the user interface member specified herein. Therefore, all features disclosed for the user interface member are also disclosed for the drug delivery device and vice versa.

According to at least one embodiment, the drug delivery device comprises a container holder for holding a drug container. The container holder may be a housing of the drug delivery device or may be connected or connectable to the housing. The container holder may be configured to hold the drug container axially and/or rotationally fixed with respect to the housing of the drug delivery device. Particularly, the container holder may hold the drug container such that the drug container does not move in axial and/or rotational direction during a drug delivery process.

According to at least one embodiment, the drug delivery device comprises a drug container filled with a drug. The drug container may be a syringe with a pre-mounted needle at a distal end. Alternatively, a needle may be attachable to the drug container, e.g. a distal end thereof.

A method for operating the drug delivery device may be as follows: The user interface member is touched or held by a user, e.g. at the lateral surface, and rotated thereby dialing a dose to be injected to the user. The user interface member may be rotated on a helical path with respect to the housing and/or the drug container (holder), thereby moving e.g. in proximal direction. After having dialed the desired dose, the user interface member may be pushed in axial direction, e.g. in distal direction, and the dose of the drug is injected. For this purpose, a user may press against the proximal surface of the user interface member. Before or after dialing the dose and injecting the dose, the user may operate the user interface member in a first way, e.g. press it in distal direction for a first time period, to initiate a first operation state, e.g. advertising for a pairing with the further device, such as to establish a Bluetooth connection/pairing. This first operation state may be indicated to the user by generating an illumination pattern assigned to the first operation state. Additionally or alternatively, the user may operate the user interface member in a second way, e.g. press it in distal direction for a second time period, to initiate a second operation state, e.g. attempting to perform or performing data synchronization of the drug delivery device with a further device, e.g. dose information. The second operation state may be indicated to the user by generating a further illumination pattern assigned to the second operation state.

Operating the user interface member in the first and/or second way may also be done without dialing a drug dose and/or injecting a dose before or after. That is to say, the first way and the second way of operating the user interface member may be used in the initial or zero dose set position.

Features described for the user interface member, the drug delivery device and the method can be combined with each other, even if the combination is not expressly disclosed herein. For example, features relating to the user interface member or the drug delivery device do also apply for the method and vice versa.

Hereinafter, the user interface member, the drug delivery device and the operation method of the user interface member described herein will be explained in more detail with reference to drawings on the basis of exemplary embodiments. Same reference signs indicate same elements in the individual figures. However, the size ratios involved are not necessarily to scale, individual elements may rather be illustrated with exaggerated size for better understanding

### BRIEF DESCRIPTION OF THE DRAWINGS

FIG. 1 shows an exemplary embodiment of a drug delivery device in an exploded view,

FIGS. 2 and 3 show proximal sections of an exemplary embodiment of the drug delivery device in different operation states,

FIGS. 4 and 5 show different views of a proximal section of an exemplary embodiment of the drug delivery device,

FIGS. 6 to 8 show exemplary embodiments of different illumination patterns.

FIG. 9 illustrates an embodiment of a user interface member on the basis of a sectional view.

FIGS. 10A to 10C illustrate another embodiment of the light distribution element.

### DETAILED DESCRIPTION

In the following, exemplary embodiments will be described with reference to an insulin injection device. The present disclosure is however not limited to such application and may equally well be deployed with injection devices that are configured to eject other medicaments or with drug delivery devices in general, preferably pen-type devices and/or injection devices.

Certain exemplary embodiments in this document are illustrated with respect to a drug delivery device in the form of an injection device where the user interface member is formed as a knob realizing an injection button and a dose setting (dialling) member at the same time, e.g. similar to the device disclosed in WO 2014/033195 A1 or WO 2014/033197 A1. Thus, the knob may be used for initiating and/or performing a dose delivery operation of the drug delivery device and may also be used for initiating and/or performing a dose setting operation. The devices may be of the dial extension type, i.e. their length increases during dose setting. Other injection devices with the same kinematical behaviour of the dial extension during dose setting and dose expelling operational mode are known as, for example, the Kwikpen® or Savvio® device marketed by Eli Lilly and the FlexPen®, FlexTouch® or Novopen® device marketed by Novo Nordisk. An application of the general principles to these devices therefore appears straightforward and further explanations will be omitted. However, the general principles of the present disclosure are not limited to that kinematical behaviour.

Certain other embodiments may be conceived for application to injection devices where there are separate injection button and grip components/dose setting members e.g. the device disclosed in WO 2004/078239 A1. Thus, the present disclosure also relates to systems with two separate user interface members, e.g. one for the dose setting operation and one for the dose delivery operation. In order to switch between a dose setting configuration of the device and a dose delivery configuration, the user interface member for dose delivery may be moved relative to the user interface member for dose setting.

If one user interface member is provided, the user interface member may be moved distally relative to a housing. In the course of the respective movement, a clutch between two members of a dose setting and a drive mechanism of the device changes its state, e.g. from engaged to released or vice versa. When the clutch, e.g. formed by sets of meshing teeth on the two members, is engaged, the two members may be rotationally locked to one another and when the clutch is disengaged or released, one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

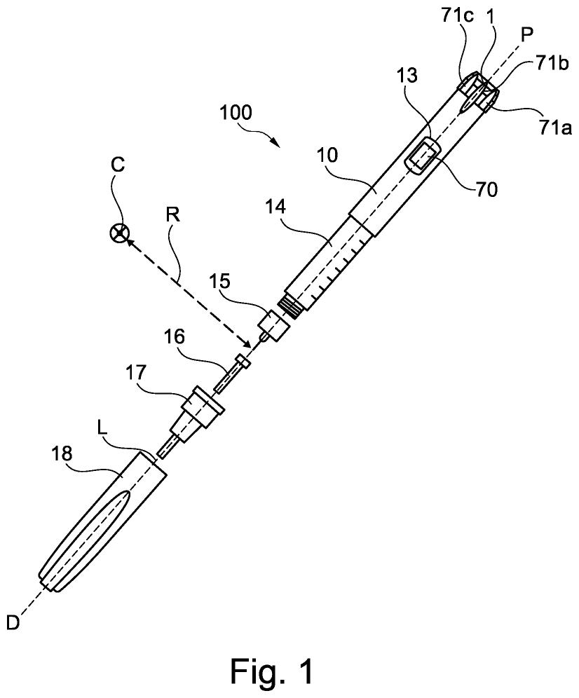

FIG. 1 is an exploded view of an exemplary embodiment of a drug delivery device 100. In this exemplary embodiment, the drug delivery device 100 is an injection device, e.g. a pen-type injector.

The injection device 100 of FIG. 1 is an injection pen that comprises a housing 10 holding a drug container 14, e.g. an insulin container, or a container holder for such a container 14. The container 14 may contain a drug, e.g. insulin. The container 14 may be a cartridge or a receptacle for a cartridge which may contain the cartridge or be configured to receive the cartridge. A needle 15 can be affixed to the container 14 or the receptacle. The container 14 may be a cartridge and the receptacle may be a cartridge holder. The needle 15 is protected by an inner needle cap 16 and either an outer needle cap 17 or another cap 18. An insulin dose to be ejected from the injection device 100 can be set, programmed, or ‘dialed in’ by turning a user interface member 1 in form of a knob 1, and a currently programmed or set dose is then displayed via dosage window 13, for instance in multiples of units. The units may be determined by the dose setting mechanism which may permit relative rotation of the knob 1 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 100 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the knob 1 is turned, to provide a visual indication of a currently programmed dose. The knob 1 is rotated on a helical path with respect to the housing 10 when turned during programming.

In this exemplary embodiment, the knob 1 includes one or more features 71a, 71b, 71c in form of formations to facilitate gripping and/or attachment of a data collection device or electronic system.

The injection device 100 may be configured so that turning the knob 1 causes a mechanical click sound to provide acoustical feedback to a user. In this embodiment, the knob 1 also acts as an injection button. When needle 15 is stuck into a skin portion of a patient, and then the knob 1 is pushed in an axial direction, the insulin dose displayed in display window 13 will be ejected from injection device 100. When the needle 15 of injection device 100 remains for a certain time in the skin portion after the knob 1 is pushed home, the dose is injected into the patient's body. Ejection of the insulin dose may also cause a mechanical click sound, which is however different from the sounds produced when rotating the knob 1 during dialing of the dose.

In this exemplary embodiment, during delivery of the insulin dose, the knob 1 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

The injection device 100 may be used for several injection processes until either the insulin container 14 is empty or the expiration date of the medicament in the injection device 100 (e.g. 28 days after the first use) is reached.

Furthermore, before using injection device 100 for the first time, it may be necessary to perform a so-called “prime shot” to ensure fluid is flowing correctly from insulin container 14 and needle 15, for instance by selecting two units of insulin and pressing knob 1 while holding injection device 100 with the needle 15 upwards. For simplicity of presentation, in the following, it will be assumed that the ejected amounts substantially correspond to the injected doses, so that, for instance the amount of medicament ejected from the injection device 100 is equal to the dose received by the user.

As explained above, the knob 1 also functions as an injection button so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting, the user interface member is moved proximally and/or rotates relative to the housing. During dose delivery, the user interface member moves axially, e.g. distally, preferably without rotating relative to the housing or body.

FIG. 1 also indicates the coordinate system used herein for specifying positions of members or elements or features. The distal direction D and proximal direction P run parallel to the longitudinal axis L. The longitudinal axis L is a main extension axis of the device 100. The radial direction R is a direction perpendicular to the longitudinal axis L and intersecting with the longitudinal axis L. The azimuthal direction C, also referred to as angular direction or rotational direction, is a direction perpendicular to the radial direction R and to the longitudinal axis L. The different directions and axes will not be indicated in every of the following figures in order to increase the clarity of the figures.

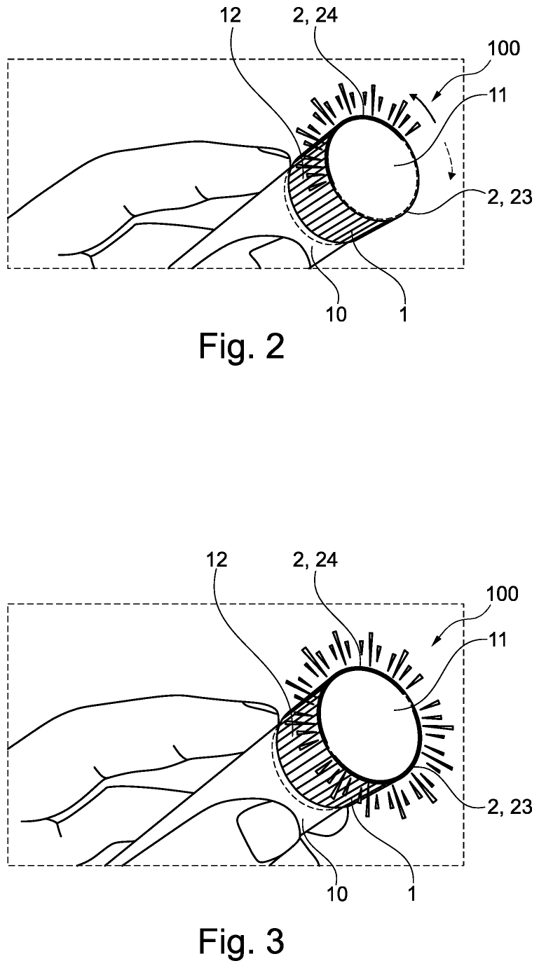

FIG. 2 shows a proximal section of the drug delivery device 100. The knob 1 has a light-emitting region 2 which comprises two subregions 23, 24. The light-emitting region 2 forms the transition region, particularly the edge, between a lateral surface formed by a gripping element 12 and a proximal surface formed by a cover element 11. The light-emitting region 2 is, e.g., formed from a transparent material, for example from a transparent plastic. The gripping element 12 and the cover element 11 may be each formed of an opaque material, e.g. a colored plastic.

FIG. 2 shows the drug delivery device 100 in a first operation state, e.g. a state in which it tries to establish a (Bluetooth) pairing with a further device, like a smart phone, smart watch or computer. The first operation state is visualized to a user by an illumination pattern comprising an alternating emission of light from the two subregions 23 and 24.

FIG. 3 shows the drug delivery device 100 in a second operation state, e.g. a state in which the drug delivery device 100 synchronizes data with a further device, e.g. a computer or smartphone. This second operation state is indicated by an illumination pattern comprising the two subregions 23, 24 emitting light simultaneously in a flashing mode, in which emission is turned on and turned off repeatedly.

The knob 1 may also be configured to initiate the two different operation states. For example, the knob 1 is configured such that when pressing the knob 1 in distal direction D for at least 0.5 seconds but less than three seconds, the second operation state is initiated. When pressing the knob 1 in distal direction for at least three seconds and less than 20 seconds, the first operation state may be initiated. The illumination patterns assigned to the operation states may be presented immediately after the respective operation state has been initiated, e.g. at most 100 ms after initiation.

Moreover, when pressing the knob 1 in distal direction for more than 20 seconds or for less than 0.5 seconds, a further operation state may be initiated, in which the knob 1 or the drug delivery device 100, respectively, is shutdown in order to save energy. For example, a power supply of one or more electronic elements of the drug delivery device is turned off or interrupted in the third operation state.

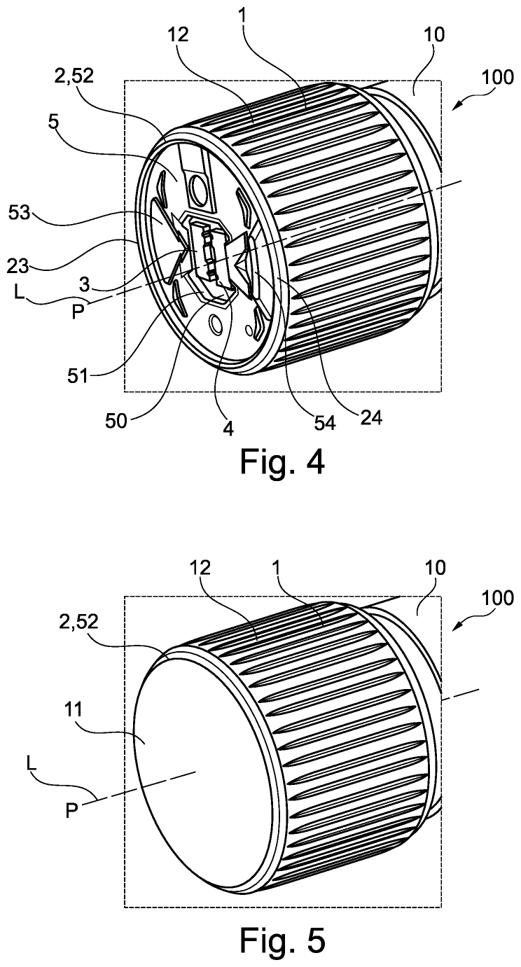

Whether the knob 1 is pressed and/or the duration how long the knob is pressed or touched may be determined via a sensor or switch. When the knob is pressed, it may be moved relative to the housing, e.g. towards the housing. Thereby, the switch may be triggered, e.g. by establishing a conductive connection or the sensor may detect that the knob is being touched or pressed. Sensor or switch signals can be evaluated by a control unit of the device (not shown in FIG. 4, e.g. a microprocessor). If it is found that the manipulation of the knob meets the conditions for one of the (pre-defined) operation states which should trigger an illumination (pattern), the control unit may issue an instruction to the light-emitting elements or LEDs 3, 4 to operate according to the pattern. Control unit and LEDs may be mounted on the same carrier e.g. a PCB.

The device may comprise an electronic dose capturing system (not shown in FIG. 4) using an encoder component and one or more sensors, e.g. an optical encoder and one or more optical sensor(s). The dose capturing system is configured to monitor and/or quantify relative movement between the encoder component and the sensor(s), e.g. via signals generated by the sensos in response to the relative movement. This relative movement may be indicative for the size of the delivered dose. An example for such a dose capturing system is disclosed in WO 2019/101962 A1, the disclosure of which is herewith incorporated by reference into the present application, in particular with respect to but not restricted to the dose capturing system.

The conditions for one of the operation states which should trigger an illumination (pattern) may comprise certain durations for which the knob is pressed (see the times mentioned further above). For example, the first and second operation states may be indicated by an illumination, where the first operation (e.g. (Bluetooth) pairing or advertising) requires a longer pressing of the knob than the second one (e.g. (dose data) synchronization). Another condition (in addition or as an alternative to the previously mentioned conditions) may be, whether the dose capturing system generates a signal, e.g. within a predetermined time, e.g. 0.5 s or less, after the button has been pressed. If a signal is detected within the predetermined time, no illumination may occur (e.g. because it is assumed that the user covers the knob with his finger for a longer time and/or is concerned with the dose delivery operation which needs no further indication as a needle pierces the user's skin already). The illumination (pattern) may only be provided if the knob is in its initial or zero dose set position (which it presumably is, if the dose capturing system does not generate signals in response to the knob being pressed).

FIG. 4 shows the proximal section of the drug delivery device 100 with the cover element 11 detached from the knob 1. The knob 1 comprises a light distribution element 5. The light distribution element 5 is formed in one piece and from a transparent material, e.g. a transparent plastic. The light distribution element 5 is disc-shaped with a main extension plane running perpendicularly to the longitudinal axis L. The light distribution element 5 comprises two subsections 53 and 54 which are assigned to the different subregions 23, 24 of the light-emitting region 2. The two subsections 53, 54 are optically separated from each other by structures in the light distribution element 5.

A recess 50 in the form of a hole is formed in the light distribution element 5. Inside the recess or hole 50, two radiation-emitting elements 3, 4 are arranged. The radiation-emitting elements 3, 4 are, e.g., LEDs, particularly so-called side lookers.

The knob 1 is configured to operate the LEDs 3, 4 depending on the operation state of the drug delivery device 100. Light emitted from the LEDs 3, 4 is coupled into a light-entry side 51 of the light distribution element 5, said light-entry side 51 or a surface thereof running perpendicularly to the main extension plane of the light distribution element 5. In the present case, the LEDs 3, 4 emit light in opposite radial directions. The light distribution element 5 may comprise two surfaces (light-entry surfaces) via which light enters the light distribution element. The surfaces may be parallel and/or face one another. The surfaces may be parallel relative to the longitudinal axis L and/or delimit a portion of the recess or hole 50 laterally.

Light which has entered the light distribution element 5 via the light-entry side 51 is guided to a light-exit side 52 of the light distribution element 5. The light-exit side 52 forms the light-emitting region 2. The light-exit side 52 is a surface of the light distribution element 5 running obliquely with respect to the longitudinal axis L and with respect to the lateral surface of the gripping element 12. Light emitted via the light-emitting region 2 is therefore directed in proximal direction P as well as in radial direction. The light-emitting region 2 or the light-exit side 52, respectively, is ring-shaped.

FIG. 5 shows the drug delivery device 100 of FIG. 4 but now with the cover element 11 attached. The cover element 11 forms the proximal surface of the knob 1. The light-exit side also runs obliquely with respect to the proximal surface formed by the cover element 11. The light distribution element and the cover element can be twin-shot molded together to form one component. This component can be clipped to the user interface member or a body thereof. Other ways of manufacturing are possible, e.g. as set out in the summary section.

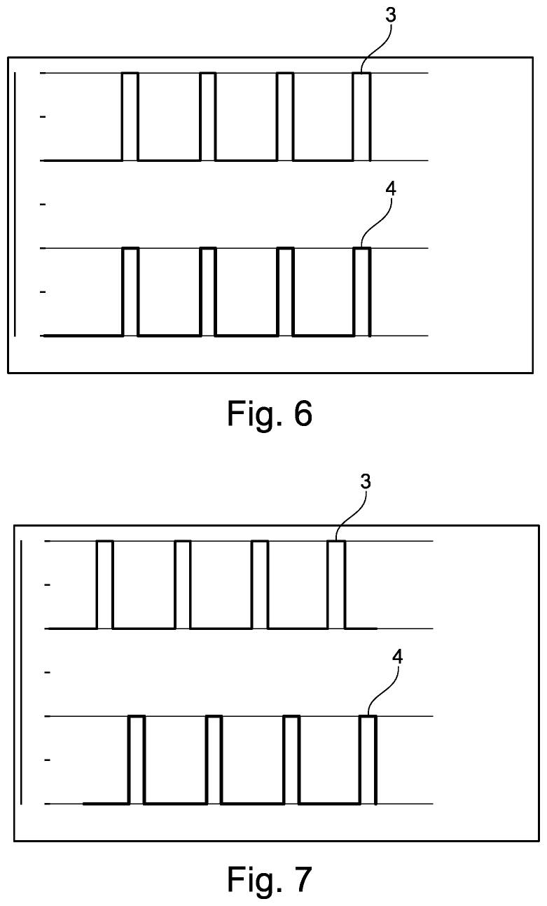

FIGS. 6 to 8 indicate different illumination patterns realizable by the knob 1. Particularly, the figures each indicate different flashing patterns of the LEDs 3, 4. Each pattern may be indicative for a particular operation state.

In FIG. 6, the LEDs 3, 4 are operated simultaneously or synchronized in a flashing mode, such that the LEDs are activated simultaneously. This may indicate that the device is advertising for conducting and/or conducting a (dose data) synchronization process. In FIG. 7, the LEDs 3, 4 are each operated in a flashing mode but in an alternating manner so that when LED 3 is activated (emits light), LED 4 is turned off (does not emit light) and when LED 4 is activated, LED 3 is turned off. The time period for which an LED 3, 4 is activated may be 100 ms and the frequency with which the LEDs 3, 4 are activated may be 1 Hz or 0.5 Hz.

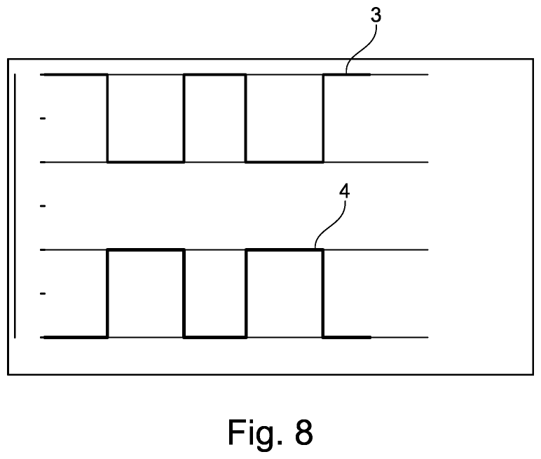

FIG. 8 shows an illumination pattern, in which both LEDs 3, 4 are operated in a flashing mode and in an alternating manner. The time windows in which the LEDs 3, 4 are activated is longer than compared to FIG. 7. This pattern may indicate that the device is advertising for pairing and/or that the pairing procedure is being conducted. Of course, further states could be indicated, e.g. a dose setting state or dose delivery state, an alarm state, etc.

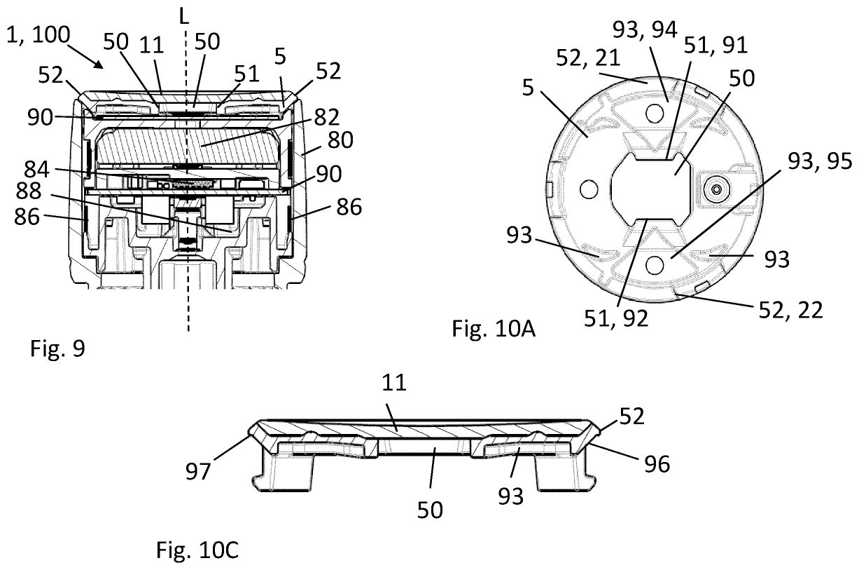

FIG. 9 illustrates an embodiment of the user interface member or knob 1 or of the drug delivery device for injection device with this knob or user interface member on the basis of a sectional view. The knob 1 has a body 80. The body 80 may provide the outer lateral or side surface of the knob which is available for manipulation by the user. Within the knob one or more electrical or electronic elements are arranged. Merely exemplarily are shown: a battery or power source 82, e.g. a coin cell; an electronic control unit 84, e.g. a microprocessor or microcontroller or an ASIC (application specific integrated circuit); one or more sensors 86, e.g. sensors suitable to measure movement between an element of a dose setting and drive mechanism of the device 100, such as radiation sensors which are sensitive to radiation reflected from a movable element which is encoded to cause changes in the radiation reflected onto the sensor when there is relative movement, the sensor(s) 86 may communicate with the electronic control unit; a sensor or switch 88, e.g. to detect whether the knob one is being pressed towards the housing and, preferably, to communicate with the electronic control unit.

The knob 1 may have according elements in the previously discussed embodiments. The other features discussed in the previous embodiments do also apply to this embodiment of the user interface member unless differences are emphasized. For example, in the proximal end region of the knob 1, the light distribution element 5 is shown. This element may be configured as previously described or as described further below. Also, the cover element 11 is shown. The light distribution element 5 and the cover element 11 close the body 80 proximally and/or are rigidly connected to the body 80, e.g. snap-fitted. The light distribution element 5 provides a light-exit side 52. As shown in FIG. 9, the light exit side is formed by or comprises a surface of the light distribution element 5, which runs obliquely relative to the longitudinal axis L and/or to the main extension direction or plane of the light distribution element. The main extension direction or plane of the light distribution element may be perpendicular to the longitudinal axis L. The light-emitting elements or LEDs 3, 4 are not explicitly shown in this representation. Nevertheless they are provided in the region of recess or hole 50 and/or of the light-entry side 51 of the light distribution element.

One or more conductor carriers 90, e.g. circuit boards such as printed circuit boards (e.g. flexible or rigid-flexible printed circuit boards) may be arranged within the user interface member or knob 1. For example, the light-emitting elements 3, 4 and the electronic control unit 84 may be arranged on the same conductor carrier, which may have a flexible region and, hence, may achieve, the configuration depicted in FIG. 9. Alternatively, the electronic control unit 84 and light-emitting elements 3, 4 can be arranged on separate carriers 90 which are, preferably, conductively connected to one another such that the electronic control 84 unit can control operation of the light-emitting elements 3, 4.

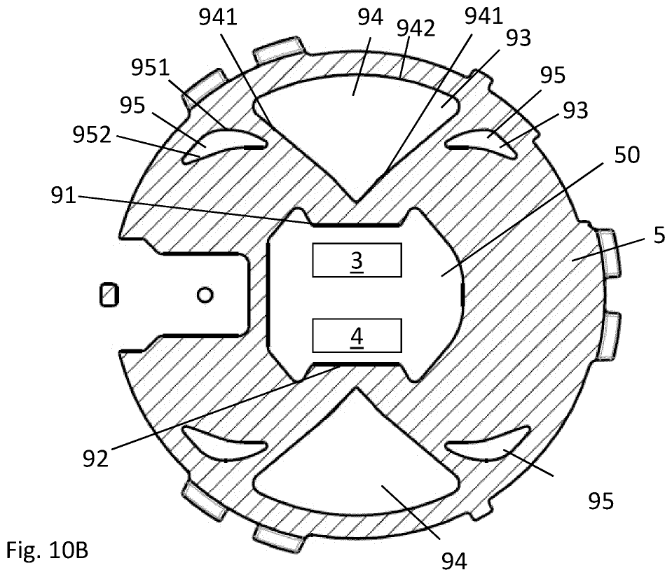

FIGS. 10A to 10C illustrate an embodiment of the light distribution element 5, e.g. the one depicted in FIG. 9. FIG. 10A shows the top view onto the light distribution element 5, especially on the proximal end thereof, which in FIG. 9 is covered by the cover element. The light-exit side 52, which may be formed ring-like and/or by a continuous surface of the light distribution element, has two subregions 21 and 22. The light-entry side 51 comprises one or more light-entry surfaces 91, 92. The surfaces 91, 92 are oppositely disposed and/or parallel to one another. The respective surface 91, 92 may be parallel to the longitudinal axis L which, in the depicted situation, is perpendicular to the plane of the representation. One light-emitting element 3, 4 may be assigned to the respective light-entry surface 91, 92.

The light distribution element 5 is configured such that light entering the light distribution element 5 through different light-entry surfaces 91, 92 is guided or directed to different subregions 21, 22. Particularly, as opposed to the light distribution element 5 discussed in the previous embodiments, the subregions 21, 22 of the light-emitting region 2 are not optically separated from one another. That is to say, optical crosstalk would, in principle be possible, where the element 5 is configured to direct the light from the light-entry surface to one associated subregion (and expediently not the other subregion). Light entering the light distribution elements 5 through the surface 91 is guided to the subregion 21 and light entering the light distribution element 5 through the surface 91 is guided to the subregion 22.

The light distribution element 5 comprises or defines one or more lights distribution systems 93. Each light-entry surface may have one associated light distribution system 93. The light distribution systems for different light-entry surfaces may be assigned to the respective light-entry surface 91, 92 positionally but otherwise be configured alike as seen along the light path from the respective light-entry surface 91 or 92 to be associated subregion 21 or 22. The light distribution system 93 is configured to spread or widen the light or light beam bundle entering through the light-entry surface. The spreading or widening may occur in a plane perpendicular to the longitudinal axis L and/or along the main radiation direction of the light-emitting elements 3, 4 or along the main extension plane of the light distribution element. Alternatively or additionally, the light distribution system 93 is configured such that light is deflected towards the light-exit side 52, e.g. by reflection and/or refraction. The deflection may result in light traveling obliquely, e.g. at an angle of 45°, with respect to the main radiation direction of the light-emitting elements 3, 4, which are parallel and/or opposite, with respect to the main extension plane of the light distribution element and/or with respect to the longitudinal axis L.

The light distribution system 93 is illustrated in FIGS. 10B and 10C in more detail. FIG. 10B illustrates a sectional view through the light distribution element 5.

FIG. 10B illustrates a light widening or spreading subsystem of the light distribution system 93. The light-emitting elements 3, 4 are shown. Each one is assigned to one of the light-entry surfaces 91, 92. The light distribution system 93, as seen from the respective light-entry surface along the light path, comprises a first or primary light distribution feature 94. The first light distribution feature 94 can be a recess or hole within the light distribution elements 5. The first light distribution feature 94 defines an air/material interface, where the material is the one of the light distribution element 5. The interface can be used to reflect or refract radiation or let radiation pass unmodified, depending on the angle of incidence on the interface. The first light distribution feature 94 can be the first feature which influences the light path within the light distribution element 5 after light has entered the element 5. The light distribution feature 94 has two (inclined) surfaces 941 and/or tapers towards the light-entry surface 91 or 92 associated with the light distribution system. The (inclined) surfaces 941 may be connected to one another in the region of the feature 94 closest to the light-entry side 91, 92 e.g. by a direct transition region. The inclined surfaces 941 may be curved, e.g. convexly curved as seen from within the feature 94 onto the respective surface. The inclined surfaces are connected by a further surface 942 which is remote from the light-entry surface 51. The surface 942 is curved, e.g. concavely curved as seen from within feature 94 onto the surface 942. The radius of curvature may be less than the radius of curvature of the light exit side in the plane perpendicular to the longitudinal axis L. The surface 942 may comprise or be formed by a segment of a circle around the longitudinal axis L. The light distribution feature 94 may have a generally triangular shape. Light which is manipulated by the light distribution feature 94 may illuminate the associated subregion 21 or 22, particularly a central portion thereof. The feature 94 may either permit the light passing through the recess associated with refractions at the interfaces or reflect radiation towards a second or secondary light distribution feature 95, e.g. by way of total internal reflection. After having passed through the light distribution feature 94, the light may re-enter the light distribution element 5 and travel further towards the light-exit side 52.

The light distribution system 93 further comprises a secondary or second light distribution feature 95, e.g. formed by a recess or hole in the element 5 with the associated interfaces (material/air). The light distribution feature 95 is expediently arranged to receive light reflected towards it by the first light distribution feature 94 and may further distribute the light in the angular direction (i.e. spread or widen the beam). One secondary light distribution feature is associated with each side of the primary or first light distribution feature 94. The light distribution feature 95 may be offset from the light distribution feature 94 in the angular direction and/or radially overlap with the light distribution feature 94, e.g. the inclined surface 941 thereof closest to the feature 95. Each of the light distribution features 95 may distribute light into an edge portion of the associated subregion 21 or 22. The central portion illuminable via the feature 94 is arranged between the edge portions which are illuminable via the light distribution features 95. The respective light distribution feature 95 has two, e.g. oppositely disposed, surfaces 951, 952. The surfaces may be differently curved, e.g. one is curved concavely and one is curved concavely. The radius of curvature may vary along the surface 951 and/or along surface 952. The surface 951 closer to the feature 94 and/or to an outer end surface of the light distribution element 5, is concavely curved as seen from within feature 95 onto the surface 951 The surface 952 more remote from the feature 94 and/or from the outer end surface of the light distribution element, is convexly curved as seen from within feature 95 onto the surface 952.

Via the light distribution system 93 a subregion of an angular extension of at least 120° of the light-exit side 52 is illuminated (i.e. the subregion 21 or 22) via the one light-emitting element 3 or 4 associated with light-entry surface 91 or 92 and the illumination can be perceived by the user. The illuminated subregion may have an angular extension of 160° or more or 180° (or more). The illuminated subregion may have an angular extension of 200° or less or 180° or less. e.g. about 180°. The light distribution element assists in light originating from different light-emitting elements being guided to different portions of the ring-like light-exit side 52 or light exit surface as seen along the main extension plane of the light distribution element 5.

FIG. 10C illustrates a light deflection subsystem of the light distribution system 93. The light deflection system may be remote from the light spreading or widening system as seen along the light path from the light-entry side 51 to the light-exit side 52. Light may be widened or spread before it reaches the light deflection system. The light deflection subsystem comprises a light deflection surface 96. The light deflection surface 96 may be configured and arranged to reflect light incident on it and, in this way, direct the light towards the light-exit side 52 were the light can exit the light distribution element. The light deflection surface 96 is oriented obliquely relative to the longitudinal axis L, to the main radiation direction of the light-emitting element(s) and/or the main extension plane of the light distribution element 5. The light deflection surface 96 may reflect light due to total internal reflection or due to a reflective coating applied to the surface at the exterior. Radiation traveling through the first light distribution feature 93 may reenter the light distribution element 5 and, thereafter, impinge on the light deflection surface 96 such that the light is deflected, e.g. by an angle of 90° due to a 45° inclination of surface 96 (relative to the longitudinal axis L, to the main radiation direction of the light-emitting element(s) and/or the main extension plane of the light distribution element) and leave the light distribution element via the light-exit side 52. The light-exit side 52 or the associated surface may be elevated relative to the recess 50 and/or the light-emitting element(s). This facilitates providing a space delimited by the light distribution element 5 which can receive the closure element 11, which may be opaque as described further above. Below the light exit side or surface, a radially oriented surface portion may be provided to form a bearing surface 97 via which the light distribution element 5 may bear on the body 80 of the user interface member or knob 1.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (SiRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The present disclosure described herein is not limited by the description in conjunction with the exemplary embodiments. Rather, the present disclosure comprises any new feature as well as any combination of features, particularly including any combination of features in the patent claims, even if said feature or said combination per se is not explicitly stated in the patent claims or exemplary embodiments.

1-15. (canceled)

16. A user interface member for a drug delivery device, comprising:
a light-emitting region comprising two or more subregions, the light-emitting region configured to be illuminated by at least two light-emitting elements to emit light from the light-emitting region and to visually indicate an operation state of the drug delivery device to a user;
wherein the two or more subregions are illuminable independently of each other by the at least two light-emitting elements to present different illumination patterns to a user via the light-emitting region; and
wherein the different illumination patterns indicate different operation states of the drug delivery device to the user.

17. The user interface member of claim 16, wherein the user interface member comprises the at least two light-emitting elements; and
wherein the user interface member is configured to operate the at least two light-emitting elements depending on the operation state of the drug delivery device to present different illumination patterns to a user via the light-emitting region.

18. The user interface member of claim 16, wherein the user interface member comprises at least two light-emitting elements, the at least two light-emitting elements emit light of the same color.

19. The user interface member of claim 18, wherein each light-emitting element of the at least two light-emitting elements is assigned to one subregion of the light-emitting region.

20. The user interface member of claim 16, wherein the different illumination patterns comprise one or more of a first illumination pattern, a second illumination pattern, a third illumination pattern, and a fourth illumination pattern;
wherein the first illumination pattern comprises light emitted simultaneously from the two or more subregions in a continuous mode or a flashing mode for a predetermined first pattern time;
wherein the second illumination pattern comprises light emitted in an alternating manner from the two or more subregions for a predetermined second pattern time;
wherein the third illumination pattern comprises light emitted from one subregion of the two or more subregions for a predetermined third pattern time in a continuous mode or flashing mode while another subregion of the two or more subregions does not emit light during the predetermined third pattern time; and
wherein the fourth illumination pattern comprises light of different brightness emitted from the two or more subregions for a predetermined fourth pattern time.

21. The user interface member of claim 16, comprising:
a cylindrical shape;
a proximal end; and
an outer surface;
wherein the light-emitting region extends circumferentially at the outer surface and has a linear shape; and
wherein the light-emitting region is formed at the proximal end.

22. The user interface member of claim 16, wherein the light-emitting region is ring-shaped.

23. The user interface member of claim 16, comprising:
a light-entry side;
a light-exit side; and
a light distribution element configured to receive light from the at least two light-emitting elements at the light-entry side and to transfer the light to the light-exit side assigned to the light-emitting region.

24. The user interface member of claim 23, wherein the light-exit side is formed by a surface of the light distribution element running obliquely to a main extension plane of the light distribution element.

25. The user interface member of claim 23, wherein the light distribution element comprises at least two subsections, wherein each subsection of the at least two subsections is assigned to one subregion of the two or more subregions of the light-emitting region, wherein the at least two subsections are optically decoupled.

26. The user interface member of claim 23, wherein a recess is formed in the light distribution element, the recess is configured to receive the at least two light-emitting elements.

27. The user interface member of claim 26, wherein the recess is a hole, and the light-entry side adjoins the recess.

28. The user interface member of claim 23, wherein the light distribution element is configured to guide light from the light-entry side to the light-exit side by reflection and/or refraction.

29. The user interface member of claim 16, wherein the user interface member is a knob configured to be rotated and/or axially moved relative to a housing of the drug delivery device when operated by a user.

30. The user interface member of claim 16, wherein the user interface member is configured to be manually operated by a user to initiate different operation states of the drug delivery device, and to generate different electronic signals depending on how the user interface member is operated;
wherein the different electronic signals are associated with different operation states of the drug delivery device; and
wherein the different operation states are assigned to the different illumination patterns.

31. The user interface member of claim 30, wherein manual operation comprises touching, pressing, and/or rotating the user interface member.

32. A drug delivery device comprising:
a user interface member comprising:
a light-emitting region comprising two or more subregions, the light-emitting region configured to be illuminated by at least two light-emitting elements to emit light from the light-emitting region and to visually indicate an operation state of the drug delivery device to a user;
wherein the two or more subregions are illuminable independently of each other by the at least two light-emitting elements to present different illumination patterns to a user via the light-emitting region; and
wherein the different illumination patterns indicate different operation states of the drug delivery device to the user; and
a container holder for holding a drug container.

33. The drug delivery device of claim 34, comprising a drug container filled with a drug.

34. The drug delivery device of claim 33, wherein the drug delivery device is an injection device.

35. A method of delivering a drug from a drug delivery device, the method comprising using the drug delivery device of claim 33.
